(19) United States
(12) Patent Application Publication
(10) Pub. No.: US 2022/0047223 A1
(43) Pub. Date: Feb. 17, 2022

(54) Virtual Patient Care (VPC) Platform Measuring Vital Signs Extracted from Video During Video Conference with Clinician

(71) Applicant: Cooey Health, Inc., Fort Myers, FL (US)

(72) Inventors: Srikanth Gondi, Sunnyvale, CA (US); Suman Puthana, San Jose, CA (US); Rajesh Kumar Rathinasamy, Allen, TX (US); Uma Mahadevan, Cupertino, CA (US)

(21) Appl. No.: 17/084,952

(22) Filed: Oct. 30, 2020

Related U.S. Application Data
(63) Continuation-in-part of application No. 29/748,891, filed on Sep. 1, 2020.
Application No. 29/748,891 is a continuation-in-part of application No. 29/750,898, filed on Sep. 17, 2020.
(60) Provisional application No. 63/066,021, filed on Aug. 14, 2020.
(60) Provisional application No. 63/082,062, filed on Sep. 23, 2020.

(51) Int. Cl. A61B 5/00; A61B 5/0205; G16H 50/20; G16H 40/67; G16H 80/00

(52) U.S. Cl. CPC A61B 5/7278; A61B 5/0077; A61B 5/0013; A61B 5/0022; A61B 5/743; A61B 5/021; A61B 5/7264; A61B 5/0205; G16H 50/20; G16H 40/67; G16H 80/00; A61B 5/7465

(57) ABSTRACT

A Virtual Patient Care (VPC) platform establishes a video call between a patient application and a clinician application. During the video call Selfie Vitals are measured using the selfie camera on the patient's smartphone. The patient's video is paused to the clinician and sent to Artificial Intelligence (AI) engines using remote Photoplethysmography (rPPG) or Transdermal Optical Imaging (TOI) techniques to extract vital signs. Pulse, blood pressure, oxygen saturation, and respiration rate vital signs are generated and displayed on the clinician' s user interface. Audio continues while video conferencing may be paused during vitals measurement. The patient's smartphone can perform pre-processing to generate metadata that is sent to a VPC server with more powerful AI engines. Vitals measurement can be initiated by the patient selecting a Selfie Vitals icon on a patient user interface during the video conference call or can be initiated by the clinician.

### RELATED APPLICATIONS

This application claims priority to the co-pending provisional applications for “Virtual Patient Care Platform and System”, U.S. Ser. No. 63/066,021, filed Aug. 14, 2020, and “Virtual Patient Care (VPC) Platform”, U.S. Ser. No. 63/082,062, filed Sep. 23, 2020, hereby incorporated by reference. This application also claims priority to the co-pending design patent applications for “Patient User Interface for Patient Measuring Vital Signs During Video Conference with Clinician”, U.S. Ser. No. 29/748,891, filed Sep. 1, 2020, and “Clinician User Interface for Patient Measuring Vital Signs During Video Conference with Clinician”, U.S. Ser. No. 29/750,898, filed Sep. 17, 2020, hereby incorporated by reference.

### FIELD OF THE INVENTION

This invention relates to a Virtual Patient Care (VPC) platform, and more particularly to contactless vital sign measurement using video during a video conference.

### BACKGROUND OF THE INVENTION

During a traditional visit at a doctor's office, the doctor, nurse, or other assistant places a blood-pressure monitor on the patient's arm and measures the patient's systolic and diastolic blood pressure. An oximeter may be clipped on to the patient's finger to measure his oxygen saturation, and his pulse may be taken manually or using these devices. These vital signs are recorded in the patient's record, usually before the doctor enters the room to talk to the patient. The doctor may adjust the patient's prescription medications or make other adjustments to the patients care routine as a result of the measured vital signs and other data.

The Covid-19 pandemic has greatly accelerated the migration to telemedicine. Doctors and many patients prefer the safety of remote visits using video conferencing tools such as Zoom. The physician may visually evaluate the patient during a videoconference call, and may ask the patient to move his camera, such as to look more closely at a skin lesion or wound. However, vital signs are not taken during the video call, so the physician is making care decisions on a reduced set of data.

The patient may have his own equipment, such as a personal blood-pressure monitor or his own pulse oximeter. The patient may be trained on how to use this equipment to take his own vital signs, so that the patient may report his vitals to the physician during or before the video conference call. However, some patients may not have access to such equipment or may be unable to accurately use the equipment and may require the assistance of a caregiver.

More recently, connected equipment is becoming available. A blood pressure monitor may have a Wi-Fi, Bluetooth, cellular, or cable network connection. Each time the patient uses this equipment to measure his own vitals, these vitals could be sent over the Internet to the patient's healthcare network to update his patient records. Even with Bluetooth and/or network connectivity the user would still need to use a medical device to take measurements.

What is desired is a Virtual Patient Care (VPC) platform that measures vital signs during a video conference call without using any medical equipment. It is desired to measure the patient's vital signs using the video-conferencing camera during a video conference call between the patient and a clinician. Contactless vital-sign measurement is desired in situ during video conference calls between a patient and a physician or other clinician. It is desired to use the patient's videoconferencing camera to measure his own vital signs in real time and immediately report the measured vital signs to the clinician during the video call. It is desired to use Artificial Intelligence (AI) to analyze video of the patient to extract vital signs using Photoplethysmography (PPG), remote Photoplethysmography (rPPG), or Transdermal Optical Imaging (TOI).

### BRIEF DESCRIPTION OF THE DRAWINGS

FIG. 1 is a clinician user interface during a video conference with a patient that has contactlessly taken his vital signs from the video stream.

FIGS. 2A-2C show a patient user interface

FIG. 3 is a flowchart of a VPC workflow measuring patient vitals during an augmented video call.

FIGS. 4A-4B show user interfaces when a video call is initiated.

FIGS. 5A-5B show user interfaces when a video call is being conducted.

FIGS. 6A-6B show user interfaces when vitals are being measured during a paused video call.

FIGS. 7A-7B show user interfaces after vitals are measured during the video call.

FIG. 8 is a block diagram of the Virtual Patient Care (VPC) platform.

FIG. 9 is a video call flow diagram.

FIG. 10 is a state diagram showing phases or states of operation of the VPC application.

FIG. 11 shows a splitter for the patient's video stream during vitals measurement.

FIG. 12 shows a switcher for the patient's video during vitals measurement.

FIG. 13 shows the clinician user interface when the patient is measuring vitals.

FIG. 14 shows another embodiment of the clinician user interface when the patient is measuring vitals.

FIGS. 15A-15C show AI engines on the patient's device and on the VPC server sharing the vitals measurement workload.

FIGS. 16A-16C show arrangements for processing to generate vital sign measurements.

FIG. 17 is a flowchart of vitals collection during an augmented video call.

FIG. 18 shows the clinician's user interface with a care team.

FIGS. 19A-19B show multiple participants on the patient user interface.

FIGS. 20A-20B show other embodiments of the patient user interface.

FIGS. 21A-21C show more variations of the patient user interface.

FIG. 22 illustrates a prior art neural network.

FIG. 23 shows training a neural network.

### DETAILED DESCRIPTION

The present invention relates to an improvement in Virtual Patient Care (VPC). The following description is presented to enable one of ordinary skill in the art to make and use the invention as provided in the context of a particular application and its requirements. Various modifications to the preferred embodiment will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed.

The inventors have realized that advanced video-processing techniques, such as Photoplethysmography (PPG), remote Photoplethysmography (rPPG), and Transdermal Optical Imaging (TOI) may be used with Artificial Intelligence (AI) to analyze the video stream from the patient's camera while on a video conference call with a clinician.

The video of the patient may be used to extract his vitals because the patient's face may subtly change as blood is pumped by his heart into his face. Pulse and blood pressure can be measured using a technique known as Transdermal Optical Imaging (TOI). Light in the visible spectrum travels beneath the skin's surface and is re-emitted before being captured by a camera sensor. TOI detects subtle changes in skin color from the difference in re-emitted light between hemoglobin and melanin chromophores to detect blood flow pulsation in the cardiovascular system.

More sophisticated AI analysis of video images of the patient's face may determine blood pressure or saturated oxygen levels or other vital signs including heart rate, heart rate variability, mental stress level (or stress index), oxygen saturation, respiration rate, or blood pressure.

The AI engine extracts vital signs based on video from the same camera on the patient's smartphone that is being used for the video call. From the patient's perspective, measurement of vitals is automatic and as simple as continuing with the video conference call.

The inventors realize that some patients, particularly older patients, may be uncomfortable with technology but have been forced to learn how to videoconference with their doctors because of the Covid pandemic. These patients are likely to have difficulty using medical equipment. The inventors realize that taking vital signs while a video conference is already in progress is advantageous for these patients, since the clinician can walk the patient through the steps to take vital signs if the video conference continues to run while taking the vital signs.

Video Stopped, Audio Continues, When Taking Vitals During Call

The inventors further realize that video conferencing suffers from limited bandwidth and processing resources that can disrupt the call with jerky video or audio gaps. To combat this limited-bandwidth and/or processing resources problem, the inventors turn off the video stream when vitals are being measured, and direct the video from the patient's camera only to the AI engines that extract the vital signs. However, the audio continues, allowing the clinician to tell the patient what to do while these vital signs are being measured. Thus the inventors solve one of the frustrating problems with video conferences that could otherwise inhibit in-call vital-sign measurement.

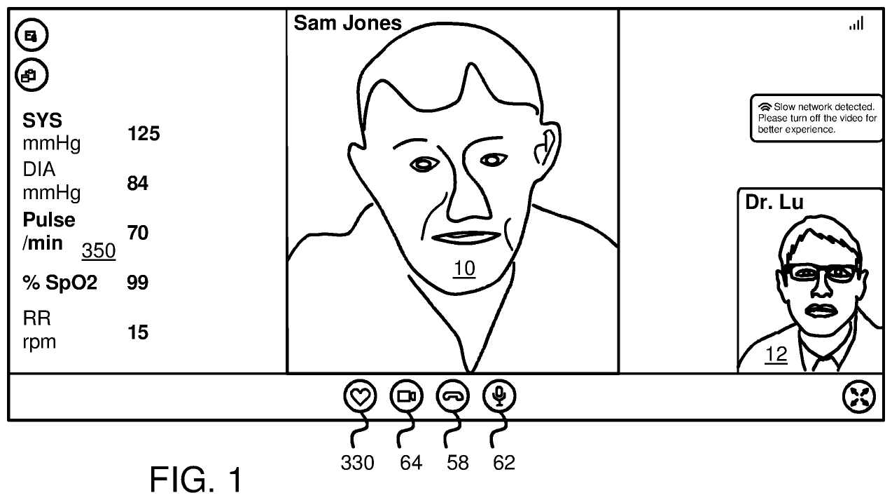

FIG. 1 is a clinician user interface during a video conference with a patient that has contactlessly taken his vital signs from the video stream. The clinician, such as a doctor, nurse, assistant, or other care provider can browse a list of patients and select a particular patient and review his records before initiating a video call with that patient. During the video call with the patient, clinician's face 12 appears in the smaller video window while patient's face 10 appears in the larger video window. Depending on the processing resources, bandwidth, and network conditions, the larger-window's video of patient's face 10 may appear smooth and crisp, or may appear jerky and blurry. A message may be displayed when poor bandwidth is detected, suggesting that the clinician turn off his video and use audio only.

Conference-controlling icons are also displayed to allow the clinician to control the video conference call. Hangup icon 58 is selected, clicked on, pressed, or otherwise activated when the clinician desires to end the call with the patient. Mute icon 62 is activated to mute the audio from the clinician during the call. Video icon 64 is activated when the clinician wants to disable his video feed to the patient or others on the call.

The clinician can press vitals icon 330 to cause the patient's vitals to be taken. Once the AI engine processes the video feed from the patient's camera to determine his vital signs, these vital sign measurements are displayed as displayed vitals 350. In this example, the patient's systolic and diastolic blood pressure, pulse, oxygen saturation SpO2, and respiration rate is displayed as displayed vitals 350.

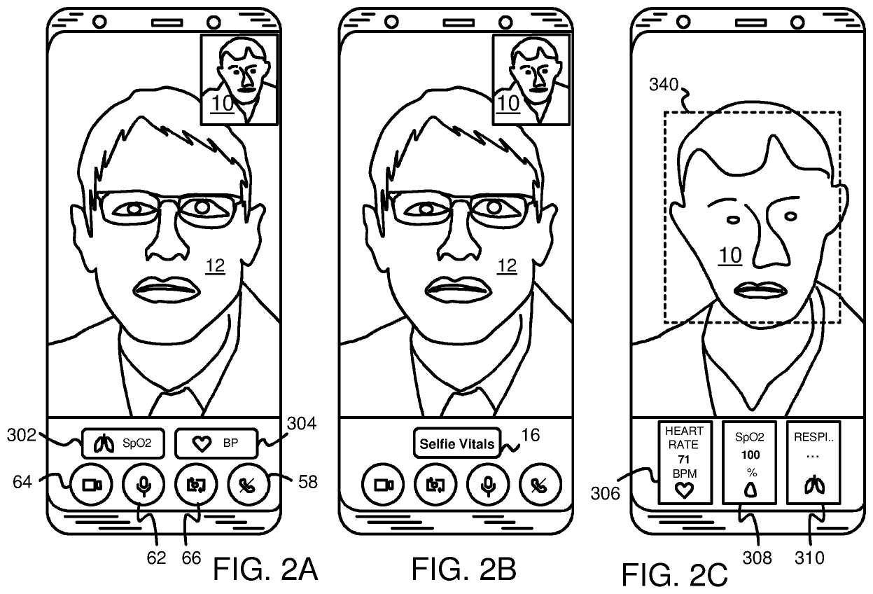

FIGS. 2A-2C show a patient user interface. The patient activates his VPC program or application (app) on his smartphone, Personal Computer (PC), tablet, smart TV, or other device, and answers the call from his clinician using the VPC app. The patient user interface is displayed on the patient's device during the video call with the clinician.

In FIG. 2A, clinician's face 12 appears in the large video window, while patient's face 10 appears in the smaller video window on the patient user interface being displayed on the patient's device. The patient can adjust or control the video call by pressing hangup icon 58 to end the call, pressing mute icon 62 to turn off his audio or microphone, and pressing video icon 64 to disable his camera or video stream. The patient may also press camera-select icon 66 to select a different camera on his device, such as switching between a selfie or backward-facing camera and a forward-facing camera on a smartphone. For example, the clinician may ask the patient to press camera-select icon 66 to switch to the forward-facing camera to take a higher-resolution image of a skin lesion.

During the video call, the clinician may ask the patient to press oxygen-saturation icon 302 to measure the patient's oxygen saturation. When oxygen-saturation icon 302 is activated, the video feed of patient's face 10 is sent to the AI engine, which analyzes the patient's video feed to determine the patient's SpO2 measurement. Also, the clinician may ask the patient to press blood-pressure measurement icon 304 to measure the patient's blood pressure, which causes the AI engine to analyze the patient's video feed to generate his measured systolic and diastolic blood pressures.

In FIG. 2B, an alternative patient user interface has oxygen-saturation icon 302 and blood-pressure measurement icon 304 replaced by a single icon. Selfie Vitals icon 16 causes the AI engine to analyze the patient's video stream and extract measurement for all supported vitals. Selfie Vitals icon 16 may be easier for the patient to understand and less intimidating than the more technical terms used by oxygen-saturation icon 302 and blood-pressure measurement icon 304.

In FIG. 2C, once the patient has pressed either oxygen-saturation icon 302, blood-pressure measurement icon 304, or Selfie Vitals icon 16, clinician's face 12 is removed and patient's face 10 is expanded to fill the larger window. Patient's face 10 is frozen in the display, or replaced by an icon or other still image. These still images require fewer processing resources of the patient's device, allowing more processing resources to be used by the AI engine that is measuring the patient's vital signs. The audio communication may continue along seamlessly in the background so as to keep the patient and clinician interaction live and to guide the patient through the vitals-measurement process.

Bounding box 340 may be displayed around patient's face 10. The AI engine examines video from within bounding box 340 and may ignore video outside of bounding box 340. The AI engine may further limit the area within bounding box 340, such as using only the cheeks and forehead of patient's face 10 to generate the oxygen saturation reading displayed on SpO2 icon 308.

Some vital signs may be extracted more quickly than others. For example, heart rate icon 306 may display the heartrate before respiration rate icon 310 is able to display the breathing or respiration rate, since the human heart rate is higher than the respiration rate. These measurements may be one-shot measurements or may be updated over time until the measurement time has finished.

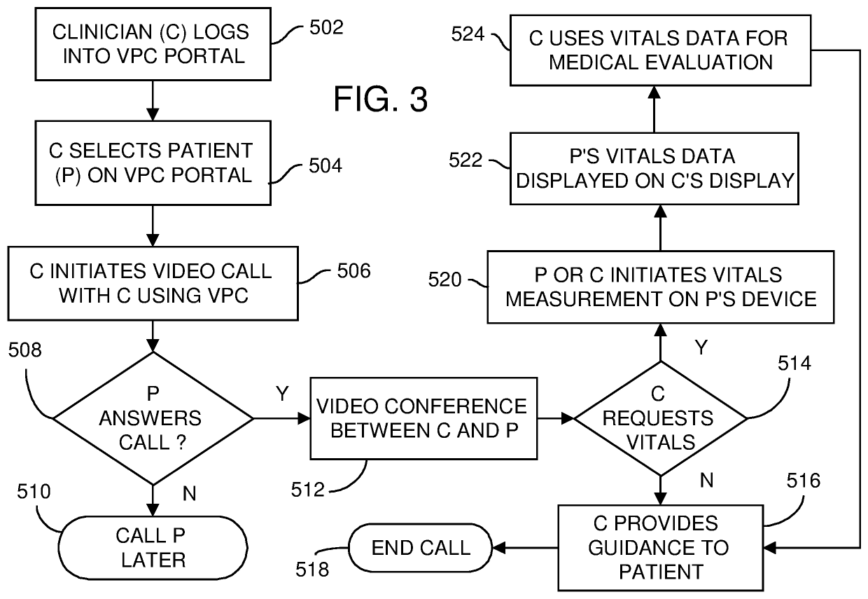

FIG. 3 is a flowchart of a VPC workflow measuring patient vitals during an augmented video call. A video call between the patient and clinician is augmented with patient information, such as his vital signs that are measured in real time during the video call. The clinician (C) logs on to the VPC portal, step 502, which could display on his office PC or workstation, or on his home PC or mobile device. A list of patients may be displayed, allowing the clinician to select a patient (P), step 504, and review his medical records and past vital sign measurements. The clinician can then initiate a video call with the patient, step 506, such as by clicking on a phone icon on the patient's record that is displayed.

When the patient does not answer the call, step 508, then the clinician can call back at a later time, step 510, and move on to another patient.

When the patient answers the video call, step 508, then a video conference is initiated between the clinician and patient, step 512. The clinician can ask questions about the patient's current health condition and any recent changes, and listen to the patient's responses to evaluate the patient's condition.

After some time of video conferencing, the clinician can request measurement of the patient's vitals, step 514. The clinician could initiate vitals measurements by pressing vitals icon 330 (FIG. 1) on the clinician's user interface, or may ask the patient to press one of his vital-measurement icons on the patient's user interface, such as Selfie Vitals icon 16 (FIG. 2B), oxygen-saturation icon 302, or blood-pressure measurement icon 304 (FIG. 2A).

Once vitals measurement is initiated, step 520, then the patient's video freezes on his device and is not sent to the clinician, but is instead sent to the AI engine to extract the vital measurements from the patient's video of patient's face 10. Once measurement is completed, the patient's vital measurements are displayed on the clinician's user interface, such as displayed vitals 350 (FIG. 1), step 522. The clinician then uses displayed vitals 350 to evaluate the patient's current medical condition, step 524, and provides consultation or guidance to the patient, step 516, before the call ends, step 518.

If the patient declines consent to measure his vitals, or of the clinician does not want to measure vitals, step 514, then the clinician can provide guidance to the patient, step 516, before the call ends, step 518.

In step 524, the clinician may determine based on the values for one or more of the measured vitals (stress index, blood pressure etc.) that there is an immediate risk for the patient's health. The clinician may intervene, step 516, by changing the patient's medicine prescription, sending a nurse or nurse assistant to the patient's home for additional diagnosis and analysis, determining that the patient needs to go to the emergency room or to urgent care, or determining that the patient needs to be called into the physician's office or hospital to meet a doctor right away. Prior-art video conferences that do not allow for real-time vital measurements could cause the clinician to miss important vitals data that would prompt the critical intervention. The patient could die.

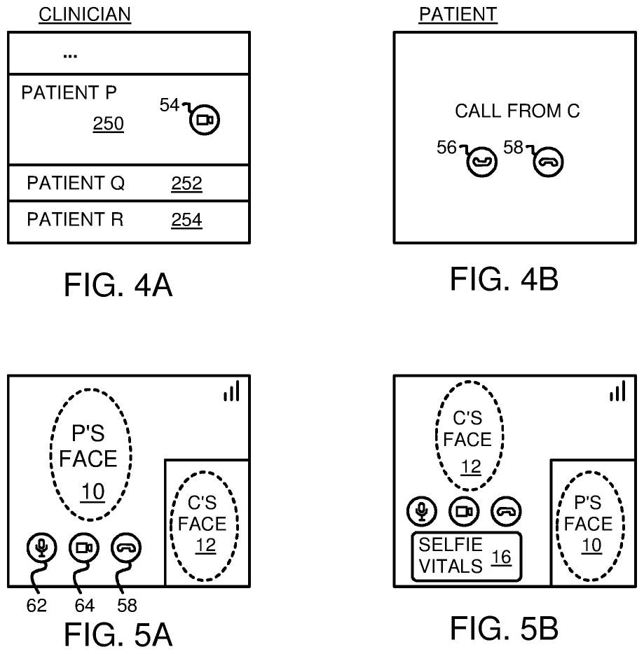

FIGS. 4A-4B show user interfaces when a video call is initiated. In FIG. 4A, the clinician user interface displays patient records 250, 252, 254 to the clinician for review. The clinician can select patient P record 250 and then clink on initiate call icon 54 to initiate a video call with patient P. In FIG. 4B, the patient user interface displays a message that an incoming call is coming from clinician C. The patient can decline this call by pressing Hangup icon 58, or can accept the call by pressing accept call icon 56.

FIGS. 5A-5B show user interfaces when a video call is being conducted. When the patient accepts the call by pressing accept call icon 56 in FIG. 4B, the user interfaces of FIGS. 5A-5B are displayed. In FIG. 5A, the clinician user interface displays patient's face 10 in a large window, and displays clinician's face 12 in a smaller window.

Conference-controlling icons are also displayed to allow the clinician to control the video conference call. Hangup icon 58 is selected, clicked on, pressed, or otherwise activated when the clinician desires to end the call with the patient. Mute icon 62 is activated to mute the audio from the clinician during the call. Video icon 64 is activated when the clinician wants to disable his video feed to the patient or others on the call. Similar conference-controlling icons are also presented to the patient in the patient user interface of FIG. 5B. However, in the patient user interface of FIG. 5B, clinician's face 12 is in the larger window and patient's face 10 is in the smaller window. Selfie Vitals icon 16 is also displayed to the patient.

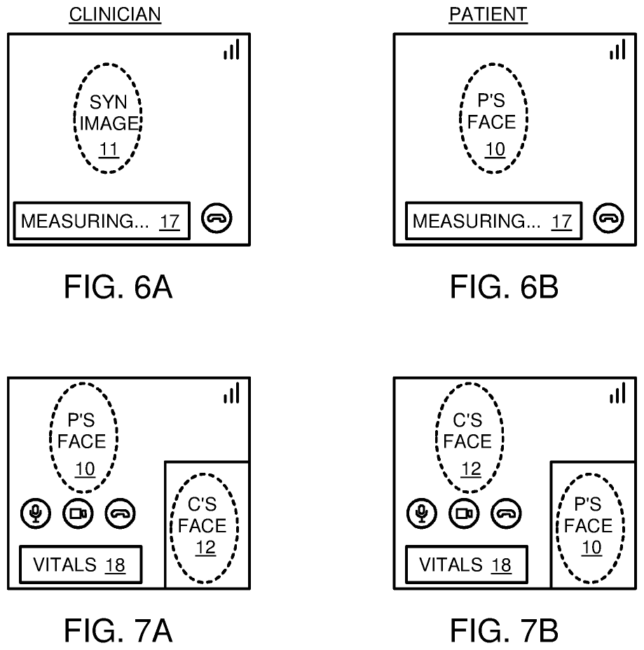

FIGS. 6A-6B show user interfaces when vitals are being measured during a paused video call. When the patient initiates vitals measurement by pressing Selfie Vitals icon 16 in FIG. 5B, the user interfaces of FIGS. 6A-6B are displayed. In FIG. 6A, the clinician user interface displays synthetic image 11 of the patient's face 10 in the large window, which can be a still image or a larger icon or other fixed display. Clinician's face 12 is no longer displayed in a smaller window, since video to and from the patient's device has been paused to allow the AI engine to have maximum processing resources to measure vitals from the patient's camera video. Measuring message 17 may be displayed on the clinician user interface to indicate that vital measurements are in progress. The clinician may still terminate the call by clicking on the Hangup icon.

In FIG. 6B, the patient user interface displays patient's face 10. The live video of clinician's face 12 is not shown while the live video of patient's face 10 is redirected to the local AI engine to allow all processing resources on the patient's device to be are used by the AI engine for vitals extraction. Measuring message 17 may be displayed on the patient user interface to indicate that vital measurements are in progress. The patient may still terminate the call by clicking on the Hangup icon. The clinician and patient may still talk to each other since audio continues when video is paused for vitals measurement.

FIGS. 7A-7B show user interfaces after vitals are measured during the video call. When the vitals measurement is completed in FIG. 6B, the user interfaces of FIGS. 7A-7B are displayed. In FIG. 7A, the clinician user interface displays patient's face 10 live in the large window, and displays clinician's face 12 live in the smaller window. Conference-controlling icons are also displayed. The newly-measured vitals are displayed as vitals display 18, such as show as displayed vitals 350 of FIG. 1. The video call is augmented with vitals measurement.

In FIG. 7B, vitals display 18 is also shown to the patient. Similar conference-controlling icons are also presented to the patient, but clinician's face 12 is in the larger window and patient's face 10 is in the smaller window. The patient and clinician can discuss the new vital measurements shown in vitals display 18 and the clinician can adjust the patients care plan. Once either the patient or clinician terminate the call, the clinician user interface reverts to that of FIG. 4A.

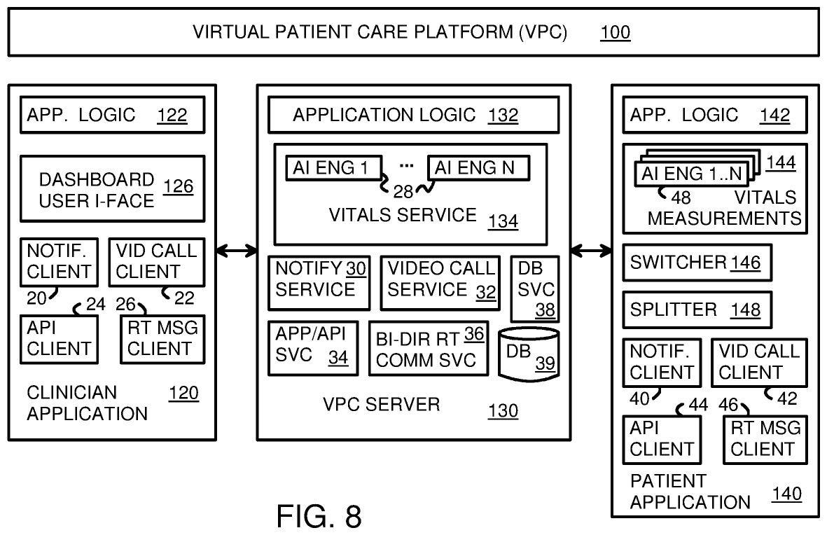

FIG. 8 is a block diagram of the Virtual Patient Care (VPC) platform. VPC platform 100 includes VPC server 130 that communicates with clinician application 120 and patient application 140.

Clinician Application 120 is a software program that is provided to the clinician to communicate and have virtual consultation and real-time vitals measurement with patients using patient application 140. Clinician application 120 has dashboard user interface 126, which enables a list or dashboard of patients and their records. Clinician application logic 122 controls other modules, such as notification client 20, real-time messaging client 26, video call client 22, and applications-programming interface (API) client 24. Real-time messaging client 26 allows the patient and clinician to interact using text messages in real time. Video call client 22 provides video calling functionality between the patient and clinician. API client 24 handles communication between clinician application 120 and the VPC Server 130.

Patient application 140 has patient application logic 142 that controls other modules, such as notification client 40, real-time messaging client 46, video call client 42, and applications-programming interface (API) client 44, that communicate with their counterparts in clinician application 120 to conduct the video conference call.

VPC server 130 has server application logic 132 that controls other blocks, such as notification service 30, bi-directional real-time communication service 36, video call service 32, and applications and API service 34, that provide communication services for their counterparts in clinician application 120 and patient application 140 to conduct the video conference call.

Database service 38 provides access to database 39 which stores persistent data. Some of the persistent data stored in database 39 may include clinician information, patient information and their health records with vitals, communication messages, tokens, identifiers, logs, configurations. Various other persistent data objects, tokens, identifiers, logs, metadata, video, and records may be stored by database 39.

When the patient initiates vitals measurements, switcher 146 or splitter 148 is activated to pause video while allowing audio to continue. The patient's video is directed instead to AI engine 48 in vitals measurement module 144. AI engine 48 may extract the vitals measurements from the local video stream, and send them to vitals service 134 in VPC server 130 using bi-directional real time communication service 36, which will store the vitals data in database 39 and send the vitals data on to clinician application 120 for display to the clinician.

Alternately, AI engine 48 may perform pre-processing of the video stream, and then send intermediate data to vitals service 134. AI engines 28 in vitals service 134 then process the intermediate data to extract the vitals measurements. Vitals service 134 may have substantially more processing resources than is available on the patient's device. AI engine 48 may have more than one AI engine.

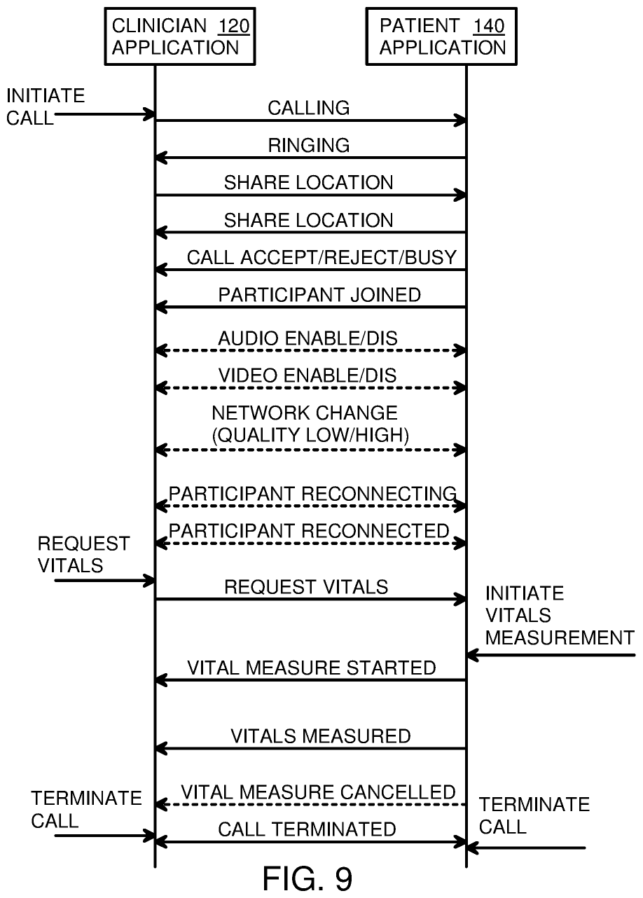

FIG. 9 is a video call flow diagram. Clinician application 120 and patient application 140 bi-directionally communicate with each other using real-time messaging clients 26, 46 (FIG. 8) by passing messages, either directly or through bi-directional real-time communication service 36 in VPC server 130 A communication protocol such as WebSocket may be used.

When the clinician decides to initiate the call, a calling message is sent from real-time messaging client 26 in clinician application 120 to real-time messaging client 46 in patient application 140, which responds back with a ringing message to indicate that the call is ringing on the patient's device, such as shown in FIG. 4B. The locations of devices for clinician application 120 and patient application 140 also may be exchanged as messages. These locations can be GPS coordinates, IP addresses, or names such as office, home, mercy hospital, etc.

If the patient is busy on another call, or rejects the call, a message is sent back to clinician application 120. If the patient accepts the call, then a call acceptance message and a participant joined message are sent back. The participant joined message can include the name of the patient, or a patient code or identifier. Video call service 32 can then facilitate the video call using video clients 22, 24 (FIG. 8). Audio can be enabled or disabled by either party clicking on mute icon 62, while video can be enabled or disabled by clicking on video icon 64 (FIGS. 1,2).

The Quality-of-Service (QoS) of the network may change over time during the video conference. This change in network quality can be indicated by a network quality change message. Sometimes a participant may be dropped from the call, either by accidentally hitting hangup icon 58 or because of network or device problems. When the participant attempts to rejoin the call, participant reconnecting and participant reconnected messages are exchanged.

After some initial discussions, the clinician may ask the patient to measure vitals, either by verbally asking the patient to click Selfie Vitals icon 16 or similar button on the patient's device, or by clicking vitals icon 330 on the clinician's device, which generates a request vitals message from clinician application 120 to patient application 140. Patient application 140 activates vitals measurement module 144 to switch off video streaming to clinician application 120 and measure vital signs from the patient's video stream. A vitals measurement started message is sent from patient application 140 when measurements are started, and a vitals measured message along with the measured vitals data is sent from patient application 140 when vital measurements have completed. If vitals measurement is cancelled or fails, a vital measurements cancelled message is sent.

The vitals are displayed to the clinician as vitals display 18, allowing the clinician to analyze the new vitals data and discuss them with the patient. Finally either the patient or the clinician clicks on hangup icon 58 and the call is terminated with call terminated messages being exchanged.

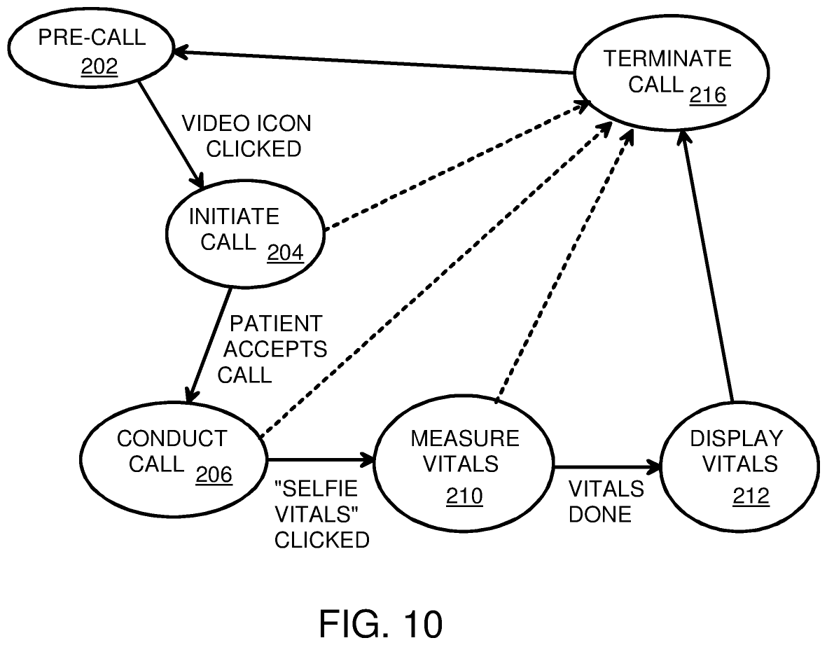

FIG. 10 is a state diagram showing phases or states of operation of the VPC application. There are 6 states. Pre-call state 202 is the state that the application is in before the clinician initiates the call. For instance, it could be the state wherein the clinician just logged in or when the clinician was performing some other activity such as reviewing records. This is also the state that the user will be taken to, after terminate state 216.

When the clinician clicks on a video call icon to initiate a video call, initiate call state 204 is entered. A RINGING event is triggered and a notification is sent to the patient application 140. A message is displayed on the clinician user interface to indicate this event. Conduct call state 206 is entered when the patient accepts the call notification and enters the video call. A CALL_CONNECTED event is generated as the video call is started. If the patient declines the call notification, terminate call state 216 is entered and a CALL_REJECTED event is triggered. A message is displayed on the clinician user interface and the video call is terminated shortly after. A CALL_EXITED or CALL_ENDED event is triggered when the patient or clinician end the call prematurely (CALL_EXITED) or at the appropriate, mutually agreed upon time (CALL_ENDED), and terminate state 216 is entered with a message is displayed on the apps and the video call is terminated shortly after. AUDIO_MUTED/AUDIO_UNMUTED or VIDEO_PAUSED/VIDEO_PLAYED events are triggered by mute icon 62 and video icon 64 buttons being pressed. The audio or video track that is sent over to the remote side is stopped/resumed. RECONNECTING/RECONNECTED events are triggered by a network condition when the clinician or patient app is disconnected and reconnected. An error message is displayed and the video call is resumed after the disruption.

When the patient clicks on Selfie Vitals icon 16, the MEASURING_VITAL event is triggered and measure vitals state 210 is entered. Patient application 140 activates vitals measurement module 144 to measuring the vitals. The video track that is sent to the clinician application 120 may be stopped and the call may enter an audio only mode.

Events for VITAL_MEASURED, VITAL_MEASURING_ERROR, or VITAL_MEASURING_CANCELED are triggered by successful, erroneous or canceled vital measurements. Both clinician and patient apps display the measurement results, an error message or a cancellation message respectively. Display vitals state 212 is entered.

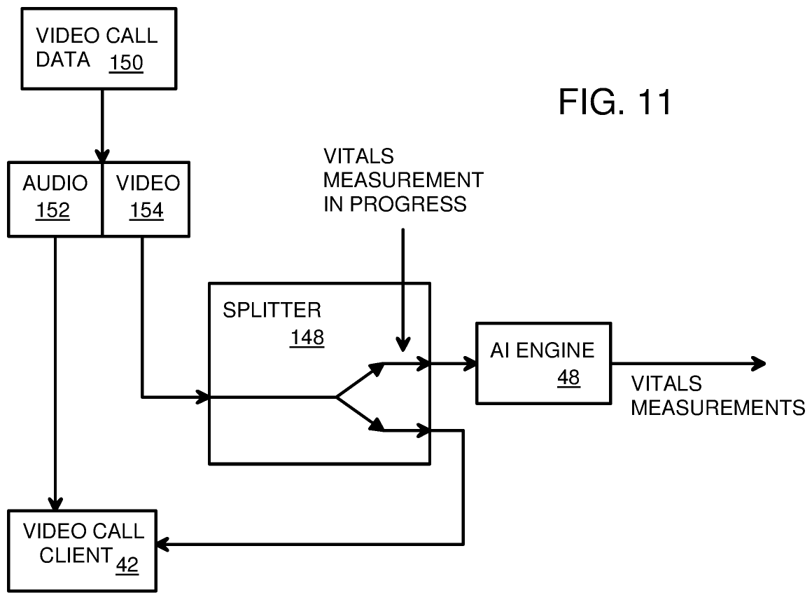

FIG. 11 shows a splitter for the patient's video stream during vitals measurement. Video call data 150 from the patient's camera is separated into audio 152 and video 154. Audio 152 is output to video call client 42 and sent to clinician application 120 so that audio can continue even when vitals measurements are taking place.

Splitter 148 splits or replicates patient's video 154 into two video streams. One video stream is sent to video call client 42 and sent to clinician application 120 so that video can continue even when vitals measurements are taking place. The other video stream is sent to AI engine 48. AI engine 48 extract vital measurements parameters from facial video based on either rPPG or TOI techniques.

Splitter 148 is useful when sufficient network or processing resources are available so that video can continue to be exchanged during vitals measurement. However, bandwidth or processing limitations are likely so using splitter 148 may disrupt vital measurement.

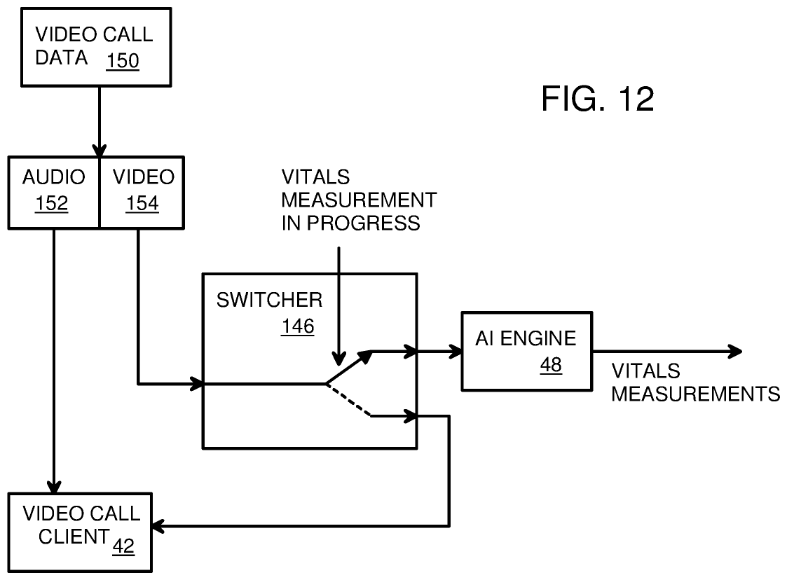

FIG. 12 shows a switcher for the patient's video during vitals measurement. Switcher 146 outputs only 1 video stream, replacing splitter 148 of FIG. 11 that outputs two video streams. When network or processing resources are limited, switcher 146 can direct these resources to vitals measurement by not sending patient's video 154 to video call client 42 when vitals are being measured. During vitals measurement, patient's video 154 is sent by switcher 146 only to AI engine 48. No video is sent to video call client 42 during vitals measurement. The network bandwidth and processing resources that would be occupied by sending patient's video 154 to clinician application 120 is saved for use by AI engine 48, which may communicate with AI engines 28 in vitals service 134 in VPC server 130 to compute the vitals. Also, any local processing resources such as video encoding or decoding used by video call client 42 to send patient's video 154 to clinician application 120 are also freed for use by AI engine 48 during vitals measurement. Thus switcher 146 frees up network and processing resources for use by vitals measurement by disabling the patient's video to the clinician.

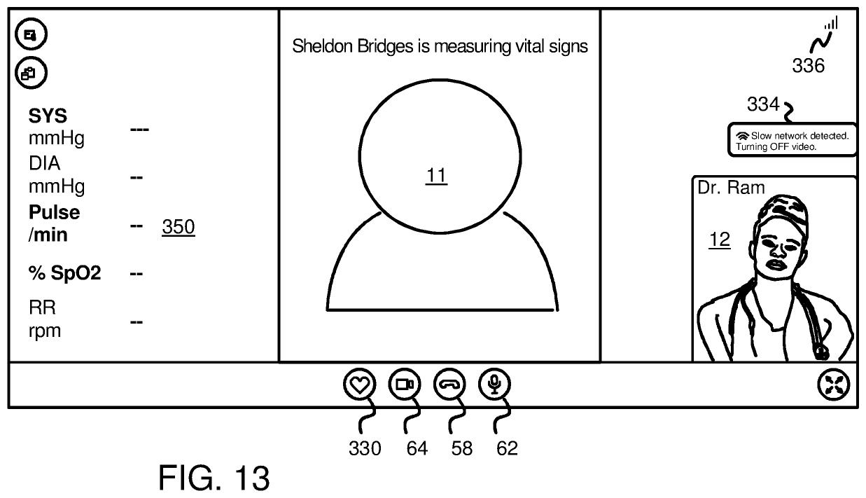

FIG. 13 shows the clinician user interface when the patient is measuring vitals. Clinician's face 12 continues to be shown back to the clinician, but patient's face 10 is no longer displayed as live video. Instead synthetic image 11 is displayed in the larger window. Synthetic image 11 can be an icon, image, or a still image of the patient's face.

Message 334 may be displayed when network quality is poor, as indicated by network strength bars 336. Dashes, blanks, dots, or other indicators may be displayed instead of the vital measurement numbers in displayed vitals 350 while vitals are being measured. A message that the patient is currently measuring vital signs can be displayed to the clinician, such as above synthetic image 11.

The vital signs being measured in this example are blood oxygen saturation SpO2, Systolic (SYS) and Diastolic (DIA) blood pressure, pulse, and Respiration Rate (RR).

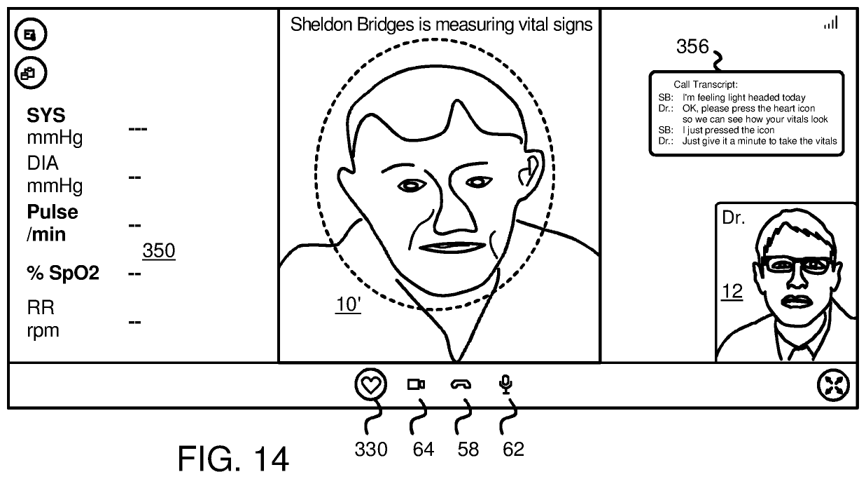

FIG. 14 shows another embodiment of the clinician user interface when the patient is measuring vitals. In this variation, a still image of patient's face 10′ is shown in the large window, such as the last video frame from patient's video 154 before switcher 146 switched the video feed to AI engine 48. Call transcript 356 is a machine-generate call transcript of the current video call. Call transcript 356 could also be a subtitle or voice transcript that is shared along with audio & video that can be rendered (or played) in synchronization with the rest of the media (Audio, video, etc.) in the session. It may be possible to use an in-band communication channel for sharing call transcript 356 during an ongoing call.

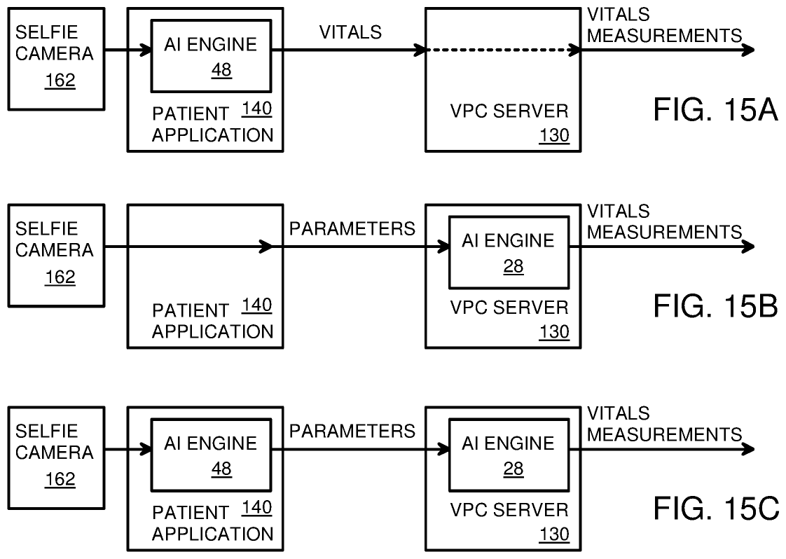

FIGS. 15A-15C show AI engines on the patient's device and on the VPC server sharing the vitals measurement workload. In FIG. 15A, AI engine 48 on patient application 140 processes patient video 154 from selfie camera 162 on the patient's device. All processing is performed locally by AI engine 48 on the patient's device.

This processing by AI engine 48 can include Artificial Intelligence (AI) to analyze video of the patient to extract vital signs using Photoplethysmography (PPG), remote Photoplethysmography (rPPG), or Transdermal Optical Imaging (TOI). In particular for TOI, each video image may be separated into 3 bitplanes for the three primary colors or red, blue, and green. Then an AI algorithm separates hemoglobin-rich (red) signals of the blood cells from the background melanin-colored signals of the skin tissue. The hemoglobin-rich signals may then be combined for all bitplanes for each video frame to generate a map of hemoglobin-rich areas of the patient's face. The changes or oscillations in these hemoglobin maps over time in the video sequence can be used to extract pulse or other vital signs. AI engine 48 may also initially detect patient's face 10 within the video, and then further detect certain facial features or areas for further processing, such as the patient's cheeks or forehead, reducing the video area that needs to be processed more fully. AI engine 48 may include a network or machine learning models that were previously trained to perform these tasks or sub-tasks, resulting in weights for node inputs within the neural network.

AI engine 48 can generate the heart rate, heart rate variability, mental stress level (or stress index), oxygen saturation, respiration rate, and blood pressure as some of the measured vital signs extracted from patient's video 154. These final vital sign measurements are sent from AI engine 48 on patient application 140 to VPC server 130 once completed.

In FIG. 15B vitals processing is offloaded to the server. The processing resources of the patient's device may be quite limited. Rather than use local processing resources, parameters from patient's video 154 may be extracted on the patient's device by vitals measurement module 144 (FIG. 8) and then these parameters are sent to VPC server 130 wherein AI engines 28 perform the bulk of the work to extract the vital measurements. In a very simple embodiment, these parameters may be the full patient's video 154, or may be facial features or blood flow data.

In FIG. 15C, vitals processing is shared. AI engine 48 in patient application 140 perform some of the earlier processing steps, such as face detection and color separation, and generate intermediate results, metadata, a mathematical model of the images, a series of images, or parameters that are sent to VPC server 130. AI engines 28 can then complete vital extraction, such as by analyzing color maps sent as parameters from patient application 140.

VPC server 130 may have many more AI engines 28 than there are for AI engine 48 on patient application 140, and AI engines 28 may be more complex, faster, or have more capabilities than AI engine 48.

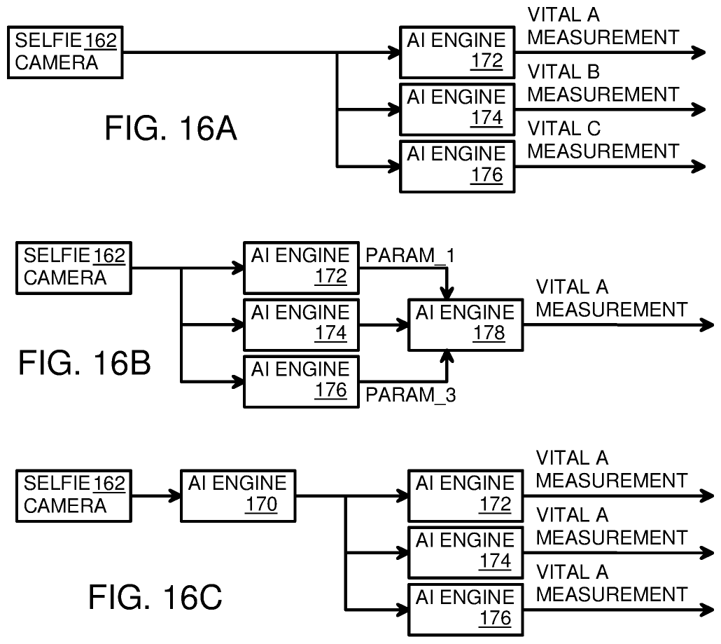

FIGS. 16A-16C show arrangements for processing to generate vital sign measurements. In FIG. 16A, patient's video 154 from selfie camera 162 on the patient's device is replicated and sent in parallel to three AI engines 172, 174, 176. These can be AI engine 48 on patient application 140, or AI engines 28 on VPC server 130, or various combinations. Each of AI engines 172, 174, 176 generates a separate vital sign independent of the other AI engines. Vital signs could also be processed serially by fewer AI engines.

In FIG. 16B, a first bank of AI engines 172, 174, 176 pre-process patient's video 154 to generate metadata or parameters that are sent to final AI engine 178 for final generation of the vital sign.

In FIG. 16C, initial processing of the patient's video is performed by AI engine 170 to generate metadata or parameters that are sent to the second bank of AI engines 172, 174, 176. The same or different parameters may be sent to each of AI engines 172, 174, 176. AI engines 172, 174, 176 then perform final generation of the vital signs. Different algorithms, weights, or configured neural networks could be used in each of AI engines 172, 174, 176 when generating the vital measurement. Three different vital signs could be generated from the three AI engines 172, 174, 176. Alternately, the same vital sign could be generated by each of the three AI engines 172, 174, 176, and then averaged together, or an outlying measurement thrown out. This can increase accuracy of the vital measurement.

Processing for different vital signs may have overlapping steps. For example, blood pressure and pulse may have the early same processing steps and then differ only in the final few steps. Using these arrangements may reduce the overall time to measure multiple vitals, thus enhancing the patient and clinician user experience.

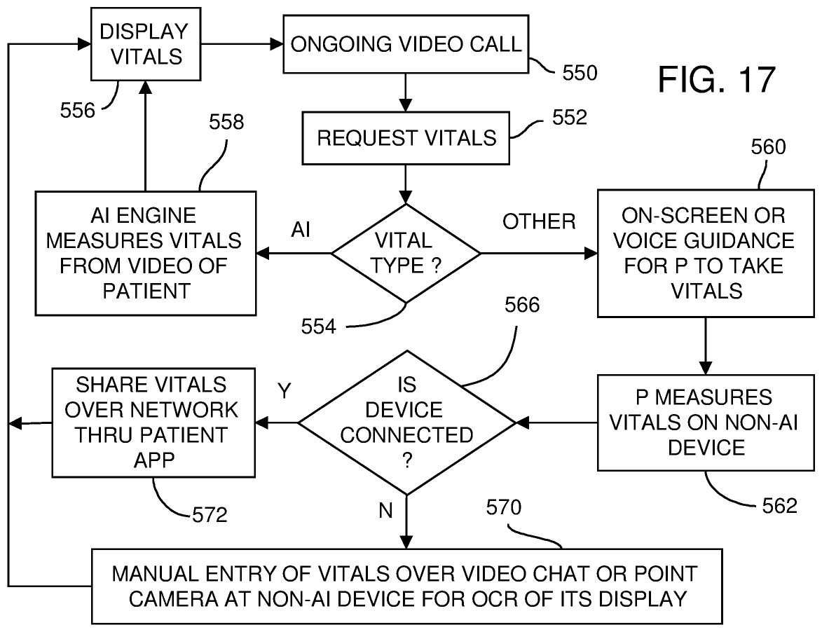

FIG. 17 is a flowchart of vitals collection during an augmented video call. During ongoing video call 550, the clinician may request that the patient's vitals be measured, step 552. Some vitals such as pulse, blood pressure, oxygen saturation, and respiration rate may be derived from the patient's video using AI engine 48. These types of vitals are referred to as AI-based vitals. When the requested vitals are AI-based vitals, step 554, then AI engine 48, 28 process patient's video 154 to generate the measured vitals, step 558. These vital-sign measurements are displayed to the clinician, step 556.

Other vital signs cannot be generated by AI engine 48 by processing patient's video 154. These types of vitals are referred to as non-AI-based vitals.

The patient may have his own external devices which do not have direct connectivity to VPC applications or platforms. Some of these external devices will have the functionality to collect the vitals but not to send the data to an application or digital solution. Such devices measure vitals, processes and show the results on a display part of the device. Normally patients write down their test results and share them with the care team.

When the vitals requested are non-AI-based, step 554, the clinician can guide the patient to use his external equipment to take his own vitals, step 560. After the patient measures his own vitals on his external equipment, step 562, then if the device is a Bluetooth or network-connected device, step 566, the patient can send these vitals from his Bluetooth or network-connected external device to VPC server 130 over the network, step 572. VPC server 130 can have a network interface added to receive such vital readings. These readings can be displayed along with the AI-based readings in displayed vitals 350.

When the external device is not a Bluetooth or network-connected device, step 566, the patient can say out loud the reading from his external device so that the clinician can hear the readings, and the clinician can enter these readings into the patient's record on the VPC platform, step 570.

Alternately, the clinician can direct the patient to point his device's camera at the display part of his external device that is showing the readings, and the clinician can type in these readings into the patient's record. A module to automatically enter the vitals measurement shown on the patient's external device may use Optical Character Recognition (OCR) of the display. For example, the patient could point his cell phone camera at a display of a blood-pressure monitor to allow OCR to capture the reading.

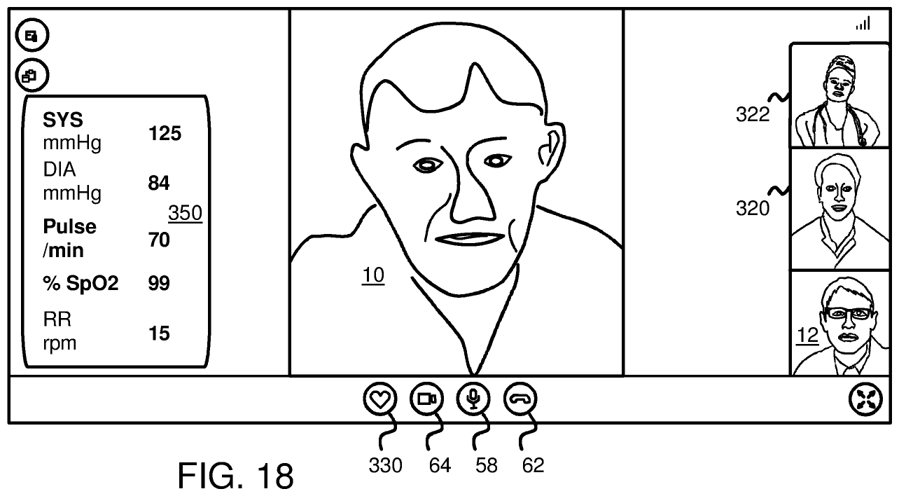

FIG. 18 shows the clinician's user interface with a care team. There may be more than two participants on a video conference call. Patient's face 10 appears in the large window while clinician's face 12 appears in the small window. Additional members of the care team appear in other live-video windows as second doctor 322 and family member 320. Displayed vitals 350 may or may not be shared with all participants.

The clinician or the patient has the option to enable multiple users to come on the call. Other users may include caregivers, family members, therapists or other members who are involved in providing care to the patient.

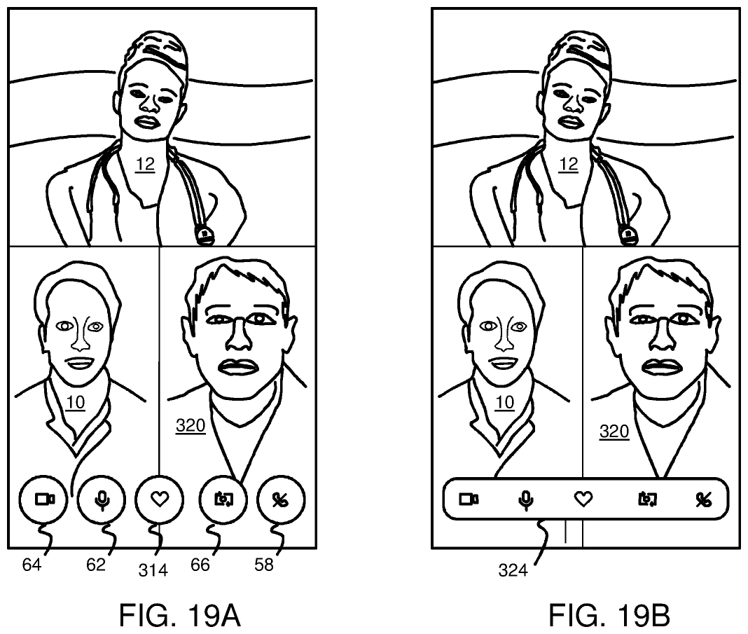

FIGS. 19A-19B show multiple participants on the patient user interface. In FIG. 19A, clinician's face 12 appears in the larger window and patient's face 10 appears in the smaller window. Video of family member 320 appears in another window.

Icons may be re-arranged or varied in appearance and function. Video icon 64, hangup icon 58, mute icon 62, camera-select icon 66, and vital-measure icon 314 can be in any order or arrangement. In FIG. 19B, icon bar 324 contains several of these icons that can be individually pressed and activated.

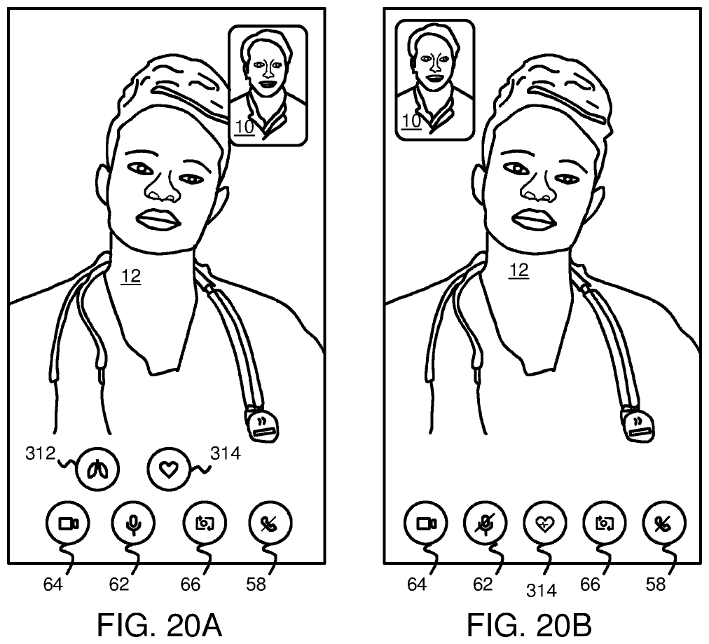

FIGS. 20A-20B show other embodiments of the patient user interface. In FIG. 20A, clinician's face 12 is displayed in the large central window while patient's face 10 is displayed in a small video window in the upper right. In FIG. 20B, patient's face 10 is displayed in the upper left. The windows may be moved around or resized in some embodiments.

Icons may be re-arranged and varied. In FIG. 20B vital-measure icon 314 in placed in the middle of the bottom row of icons, but in FIG. 20A vital-measure icon 314 is moved above the row of icons and is used only for pulse and blood pressure measurements. Another respiration vital-measure icon 312 is used to measure respiration and oxygen saturation.

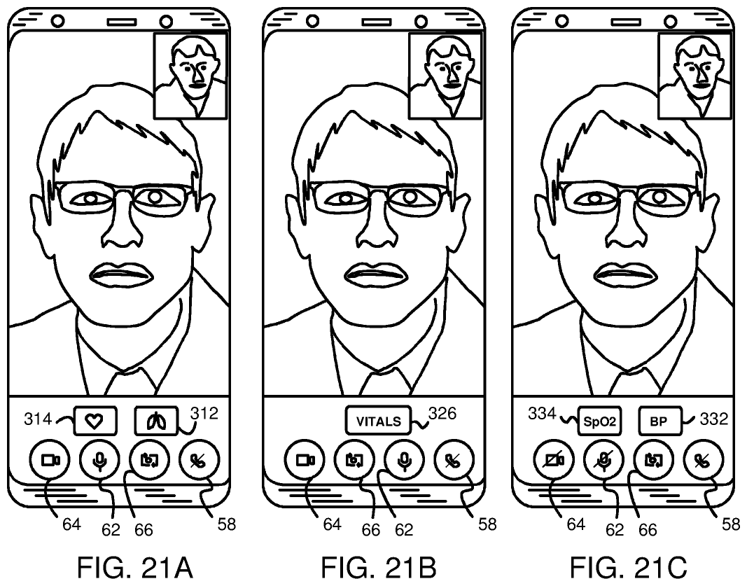

FIGS. 21A-21C show more variations of the patient user interface. In FIG. 21A, heart vital-measure icon 314 and respiration vital-measure icon 312 are in a row above hangup icon 58, mute icon 62, video icon 64, and camera-select icon 66. In FIG. 21B, a single vitals icon 326 activates all vitals measurements. In FIG. 21C SpO2 vital-measure icon 334 and BP measure icon 332 use text rather than images to indicate which vitals are measured when the icons are pressed.

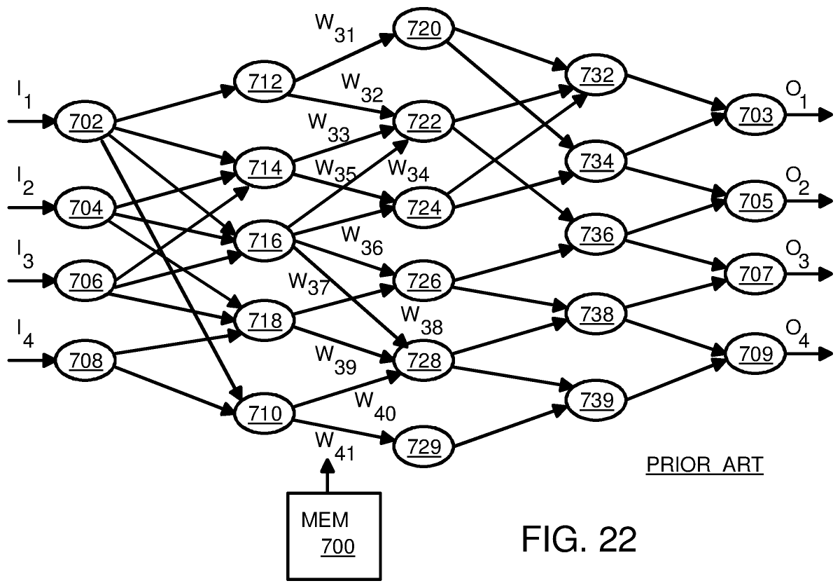

FIG. 22 illustrates a prior art neural network. AI engines 28, 48 can include a neural network such as shown herein. Input nodes 702, 704, 706, 708 receive input data I₁, I₂, I₃, . . . I₄, while output nodes 703, 705, 707, 709 output the result of the neural network's operations, output data O₁, O₂, O₃, . . . O₄. Three layers of operations are performed within this neural network. Nodes 710, 712, 714, 716, 718, each take inputs from one or more of input nodes 702, 704, 706, 708, perform some operation, such as addition, subtraction, multiplication, or more complex operations, and send and output to nodes in the second layer. Second-layer nodes 720, 722, 724, 726, 728, 729 also receive multiple inputs, combines these inputs to generate an output, and sends the outputs on to third-level nodes 732, 734, 736, 738, 739, which similarly combine inputs and generate outputs.

The inputs at each level are typically weighted, so weighted sums (or other weighted operation results) are generated at each node. These weights can be designated W₃₁, W₃₂, W₃₂, W₃₃, . . . W₄₁, etc., and have their values adjusted during training. Through trial and error or other training routines, eventually higher weights can be given for paths that generate the expected outputs, while smaller weights assigned to paths that do not generate the expected outputs. The machine learns which paths generate the expected outputs and assigns high weights to inputs along these paths. These weights can be stored in weights memory 700.

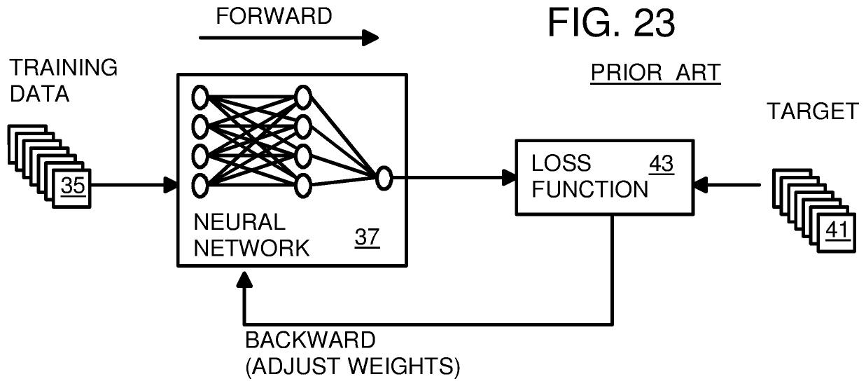

FIG. 23 shows training a neural network. Neural network 37 receives training data 35 and a current set of weights and operates on training data 35 to generate a result. The generated result from neural network 37 is compared to target data 41 by loss function 43, which generates a loss value that is a function of how far the generated result is from the target. The loss value generated by loss function 43 is used to adjust the weights applied to neural network 37. Many iterations of weights may be applied by loss function 43 onto training data 35 until a minimum loss value is identified, and the final set of weights used. AI engines 28, 48 can have a neural network that is trained with video of many different faces as training data 35, and the vital signs measured by standard equipment (blood pressure monitor, oximeter, etc.) while this video was taken as target data 41.

### Alternative Embodiments

Several other embodiments are contemplated by the inventors. Some embodiments may not use all components. Additional components may be added. Various combinations of transforms or pre-processing functions may also be substituted.

AI engines 28, 48 and their neural networks, and other components may be implemented in a variety of technologies, using various combinations of software, hardware, firmware, routines, modules, functions, etc. A trained neural network with the final weights can be implemented in an Application-Specific Integrated Circuit (ASIC) or other hardware such as FPGA's to increase processing speed and lower power consumption. Many variations are possible for training routines that operate the neural network. Optimization may first determine a number of hidden or intermediate levels of nodes, then proceed to optimize weights. The weights may determine an arrangement or connectivity of nodes by zeroing some weights to cut links between nodes. The sparsity cost may be used for initial cycles of optimization when structure is optimized, but not for later cycles of optimization when weight values are being fine-tuned. Weights, inputs, encoded weights, or other values may be inverted, complemented, or otherwise transformed. A signal processing block rather than a neural network may be used by AI engines 28, 48 to determine a vital sign. In some embodiments a combination of signal processing and neural network or machine learning models may be used.

AI engines 28, 48 may extract vital signs by detecting time-based variations or time-based characteristics of hemoglobin concentrations, such as for TOI-based extraction. rPPG-based extraction may also be used. Photoplethysmography commonly has some form of contact with the human skin, while remote photoplethysmography determines physiological processes such as blood flow without skin contact. This is achieved by using the video of the patient's face to analyze subtle momentary changes in the patient's skin color which might not be detectable to the human eye. Such camera-based measurement of blood oxygen levels provides a contactless alternative to conventional photoplethysmography. For instance, it can be used to monitor the heart rate of newborn babies or analyzed with deep neural networks to quantify stress levels.

The video of patient's case 10 may be a video of hemoglobin concentration changes that represents facial blood flow oscillations. A density mapping of hemoglobin underneath the skin keeps changing in a periodic way which relates to the oscillatory feature of the blood pressure. The color signals are indicators of this change.

Various blocks such as modules, applications, or processors may be implemented by a Central Processing Unit (CPU), or specialized processors, executing program code or firmware, or may be implemented in hardware logic gates or neural networks. Various combinations are possible. A vitals measurement processor in patient application 140 may include AI engine 48 and other components in vitals measurement module 144, and may also include AI engines 28 and vitals service 134, or may use resources in vitals service 134 or in other remote locations. VPC server 130 could be distributed across many physical server devices at different locations. AI engines 28 likewise could be distributed and could be on a different server than VPC server 130, or could be a service used by VPC server 130.

Some icons may appear or disappear for various reasons. For example, vitals icon 330 on the clinician's user interface may appear only when the patient has given prior written authorization to the medical office for taking his vitals remotely, and when the patient lives in a state or jurisdiction that requires such consent. When consent has not been given, vitals icon 330 can be removed, and the clinician can ask the patient to press Selfie Vitals icon 16 on the patient's device to initiate vitals measurement.

The clinician may first select which vitals to obtain, and then a button, icon, or message may pop up on the patient's phone, permitting the patient to take his own vitals by pressing the newly-appearing vitals buttons on his phone. For example, Selfie Vitals icon 16 might not be visible to the patient until the clinician selects which vitals to measure, and then causing Selfie Vitals icon 16 to appear on the patient's device.

In one embodiment, VPC server 130 or patient application 140 may determine that there is an insufficient lighting condition for the patient to take a reliable measurement. The VPC may provide appropriate feedback suggesting that the patient move to a better lighting environment. Under low lighting conditions, the patient's device may be enabled to automatically enable the flash of the mobile device to improve the lighting condition. The patient or doctor could be notified of insufficient lighting and the patient asked to turn on more lights or open window shades.

While the patient's face and clinician's face have been described as being shown in windows, there may be no box border around these faces, or the window border may be invisible. The window could have various shapes, such as rectangular, circular, multi-sided, fuzzy, cloud-like, etc. The window could be a view. Likewise, icons could have a circular or rectangular border or no border at all.

When vital signs are being measured, a live video of patient's face 10 may be shown back to the patient on the patient's device, such as shown in FIG. 6B, or a frozen image may be shown, or synthetic image 11, an icon, or some other image or text. For example, still image of patient's face 10′ in FIG. 14 could be replaced by patient's face 10, synthetic image 11, a textual message, or some other image, or may be completely removed. The clinician's device may show synthetic image 11, as shown in FIG. 6A, a live image of patient's face 10, a frozen image, an icon, or some other image or text while vitals are being measured.

In one embodiment, the vital signs data from the call will only be visible to selected members of the call, if more than two members are there on the call. For example, the vitals' data from the patient can be made visible only to the doctor or clinician and not to anybody else.

In another embodiment the clinician not only selects a vital sign to measure but also the AI engine(s) to use for a specific measurement. Clinician application 120 can be a stand-alone application running on the clinician's device, or it can be a web app running on a server with a Clinician user interface displayed on the clinician's device.

In one embodiment, the vital signs' data collected from the video call becomes part of the medical record of the patient and may appear almost immediately on the Clinician's portal or mobile device. The new vital-sign measurements taken on the current video call may be displayed to the clinician individually as numbers, or may be displayed graphically, such a new measurement added to a graph of past measurements to indicate trends over time and where the current measurement is compared with last measurements.

In one embodiment, the patient's camera may provide the video for two different AI engines in a sequential manner. For example, a first video sample is sent to the first AI engine, and a second video sample is sent to the second AI engine in a time sequenced manner. The first AI engine may only measure SpO2 whereas the second AI engine may measure blood pressure. Many other variations are possible.

While analysis of the patient's video to extract vital signs such as pulse, blood pressure and saturation have been described, the patient's audio could also be analyzed by AI engine 48 to detect breathing sounds to determine the patient's respiration rate. The respiration rate generated from audio could be compared to the respiration rate generated from video as a check.

A combination of switcher 146 and splitter 148 may be used for different media streams (audio, video). For example, a vital measurement that involves using both audio and video can use switcher 146 for video and a splitter for audio.

The platform may encourage patients to follow the prescribed routine with regular reminders. Reminders or notification may include notification via SMS (Short Messaging Service), mobile application platform's, push notification or email.

The patient's application or device screen may be shared over the live video call and can be used for interactive tasks. Live interactive tasks may be initiated by the clinician during the augmented video call to measure certain health signs. For example, an interactive session measures the patient's motor or cognition using on screen tasks like clicking on an object that shows on the patient's user interface. Certain vitals related to vision may be measured by altering the size of the text or size of an object shown on the patient's display. The patient can fill a form that is available either through the app or through a link in the video call interface. This enables the user to fill out a form live while on the call with the clinician. In one embodiment, the vitals measured are aggregated with the inputs on the form to determine overall health condition and possibly generate a health score. A score may be calculated based on the answers provided in the form. Based on the score and/or results provided, the clinician or the caregiver may provide appropriate clinical guidance. The clinician could use a combination of data points from the vitals information, prior medical record, answers provided in forms that are filled during the call, and interpretation of patients' visual condition during the call to make clinical decisions.

The video call service may be built on top of Internet Protocols (IP). IP calls can use Peer-to-Peer technology or be made through a centralized call server on the cloud. Peer-to-Peer connection technology enables a real time streaming between two endpoints. In a VPC case, it will be between Patient and Clinician. A call server on cloud enables real time streaming between multiple endpoints. Under VPC, this method may enable the platform to support multi party calls involving the patient, clinician and care takers or designated family members of the patient. The group of people are collectively referred to as the care team.

The concept of Augmented Video call is not limited with the type of communication used. The VPC platform may be smart enough to adapt to low bandwidth and resource availability. The video call can switch to audio only mode and still maintain the live vital measurement capability without impacting the overall care session.

Another embodiment where a call is interrupted due to low bandwidth, network failure or other issues on the patient's side, the vital measurement may be continued offline and submitted when conditions suitable for communicating with server restores. The patient could also use patient application 140 to take AI-based vitals measurements off-line, and have his app update his medical records with the new vitals measurements.

In a typical physical visit to a clinic, the clinician first takes the vitals of the patient before the doctor does the consultation. However, in telehealth video conferencing, this key aspect is missing. This invention can emulate a telehealth visit that is as close to a physical visit as possible. Conventional telehealth solutions miss out this feature. Conventional telehealth solutions, by missing out measuring the key vitals of a patient during the consultation, may miss out on key health indicators.

The background of the invention section may contain background information about the problem or environment of the invention rather than describe prior art by others. Thus inclusion of material in the background section is not an admission of prior art by the Applicant.

Any methods or processes described herein are machine-implemented or computer-implemented and are intended to be performed by machine, computer, or other device and are not intended to be performed solely by humans without such machine assistance. Tangible results generated may include reports or other machine-generated displays on display devices such as computer monitors, projection devices, audio-generating devices, and related media devices, and may include hardcopy printouts that are also machine-generated. Computer control of other machines is another tangible result.

Any advantages and benefits described may not apply to all embodiments of the invention. When the word “means” is recited in a claim element, Applicant intends for the claim element to fall under 35 USC Sect. 112, paragraph 6. Often a label of one or more words precedes the word “means”. The word or words preceding the word “means” is a label intended to ease referencing of claim elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC Sect. 112, paragraph 6. Signals are typically electronic signals, but may be optical signals such as can be carried over a fiber optic line.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

We claim:

1. A Virtual Patient Care (VPC) computing platform comprising:
a clinician application having a clinician video call client for sending real-time video of a clinician and real-time audio of the clinician during a video call with a patient;
a clinician user interface for displaying a real-time video of a patient's face to the clinician during the video call with the patient, and for playing a real-time audio from the patient;
a patient application having a patient video call client that sends to the clinician application the real-time video of the patient's face captured by a selfie camera on a patient device running the patient application, and sends the real-time audio of the patient;
a patient user interface that displays real-time video of the clinician sent from the clinician video call client and that plays the real-time audio of the clinician; and
a vitals measurement processor that processes the real-time video of the patient's face using Transdermal Optical Imaging (TOI) to generate a vital sign measurement that is captured during the video call;
wherein the clinician user interface displays the vital sign measurement to the clinician during the video call after the vitals measurement processor generates the vital sign measurement from the real-time video of the patient's face during the video call.

2. The VPC computing platform of claim 1 further comprising:
a switcher in the patient application that switches the real-time video of the patient's face to the vitals measurement processor and blocks the patient video call client from sending the real-time video of the patient's face to the clinician application during vitals measurement;
wherein the real-time audio of the clinician continues to play on the patient user interface during vitals measurement while the real-time video of the clinician is not displayed on the patient user interface during vitals measurement;
wherein the real-time audio of the patient continues to play on the clinician user interface during vitals measurement while the real-time video of the patient's face is not displayed on the clinician user interface during vitals measurement;
whereby processing resources are reserved for the vitals measurement processor during vitals measurement by not sending real-time video to the clinician application.

3. The VPC computing platform of claim 2 wherein the vitals measurement processor comprises an Artificial Intelligence (AI) engine having a neural network trained to process facial images to detect changes in color signals that indicate hemoglobin concentration, the vitals measurement processor detecting oscillations in the color signals and generating the vital sign measurement from the oscillations detected.

4. The VPC computing platform of claim 3 wherein the vitals measurement processor comprises a plurality of AI engines including a first AI engine having a first neural network trained for generating a first vital-sign measurement, a second AI engine having a second neural network trained for generating a second vital-sign measurement, and a third AI engine having a third neural network trained for generating a third vital-sign measurement, wherein the first, second, and third AI engines operate in parallel.

5. The VPC computing platform of claim 3 wherein the vitals measurement processor further comprises a plurality of AI engines including a pre-processing AI engine that receives the real-time video of the patient's face and generates metadata that is input to other AI engines in the plurality of AI engines that generate the vital sign measurement.

6. The VPC computing platform of claim 3 wherein the vitals measurement processor further comprises a pre-processor located on the patient device and an AI engine on a remote server,
whereby vitals measurement processing is distributed among the patient device and the remote server.

7. The VPC computing platform of claim 6 wherein the plurality of AI engines generate a plurality of the vital sign measurement that include a pulse rate, systolic blood pressure, diastolic blood pressure, blood oxygen saturation, and respiration rate.

8. The VPC computing platform of claim 2 wherein the vitals measurement processor further comprises a processor that performs Photoplethysmography (PPG), remote Photoplethysmography (rPPG), or Transdermal Optical Imaging (TOI) on the real-time video of the patient's face to generate the vital sign measurement.

9. The VPC computing platform of claim 8 further comprising:
a VPC server on a remote node on a network, the VPC server having a video call service for coordinating the clinician video call client and the patient video call client when establishing the video call;
a plurality of AI engines for use by the vitals measurement processor;
a database for storing persistent objects including a patient record for the patient, the patient record being augmented with the vital sign measurement generated by the vitals measurement processor.

10. The VPC computing platform of claim 8 wherein the patient user interface further comprises a vitals measurement icon that causes the vitals measurement processor to initiate vitals processing in response to the patient selecting or activating the vitals measurement icon.

11. The VPC computing platform of claim 8 further comprising:
a third-party participant application having a third video call client for sending real-time video of the third-party participant and real-time audio of a third-party participant during a video call with the patient and with the clinician; and
a third-party participant user interface for displaying a real-time video of the patient's face to the third-party participant during the video call with the patient and clinician, and for playing a real-time audio from the patient and from the clinician.

12. A computer-assisted method for taking selfie vitals during a video call comprising:
initiating a video conference call between a clinician using a clinician application running on a clinician device and a patient using a patient user interface on a patient's device;
sending video and audio from the patient's device to the clinician application, and sending video and audio from the clinician application to the patient user interface on the patient's device during the video conference call when vital signs are not being measured;
initiating vital sign measurement during the video conference call when a vital-measurement icon is selected by the patient on the patient user interface, or when the clinician initiates vital sign measurement using the clinician application;
sending audio but not sending video from the patient's device to the clinician application, and sending audio from the clinician application to the patient user interface on the patient's device during the video conference call when vital signs are being measured;
sending video of a patient's face captured from a selfie camera on the patient's device to an Artificial Intelligence (AI) engine having a neural network trained to detect blood flow oscillations in the patient's face, the AI engine generating a vital sign measurement from the blood flow oscillations; and
displaying the vital sign measurement generated by the AI engine to the clinician using the clinician application, permitting the clinician to use the vital sign measurement taken during the video conference call to medically evaluate the patient while still on the video conference call with the patient;
whereby the vital sign measurement is generated from the patient's face captured by the selfie camera during the video conference call between the patient and the clinician.

13. The computer-assisted method of claim 12 further comprising:
using a plurality of AI engines to generate a plurality of the vital sign measurement that include a pulse rate, systolic blood pressure, diastolic blood pressure, blood oxygen saturation, and respiration rate.

14. The computer-assisted method of claim 12 further comprising:
using Optical Character Recognition (OCR) to capture an external vital sign measurement generated by an external medical device, wherein the patient captures an image of a display of the external medical device using a camera on the patient's device;
displaying the external vital sign measurement to the clinician using the clinician application during the video conference call with the patient;
whereby the external vital sign measurement from the external medical device is captured by the patient's device and converted by OCR for use by the clinician.

15. The computer-assisted method of claim 12 further comprising:
displaying to the clinician the patient's face in real time in a large video window generated by the clinical application during the video conference call when vital signs are not being measured;
displaying to the clinician a synthetic image in the large video window generated by the clinical application during the video conference call when vital signs are being measured;
displaying on the patient's device the clinician's face in real time in a large video window generated by the patient user interface during the video conference call when vital signs are not being measured; and
displaying on the patient's device an image of the patient's face in the large video window generated by the patient user interface during the video conference call when vital signs are being measured.

16. A computer-program product comprising:
a computer-usable medium having computer-readable program code means embodied therein for augmenting a video call with real-time vital-sign measurements, the computer-readable program code means in the computer-program product comprising:
a patient user interface that displays a real-time video of a clinician in a large window, and displays a real-time video of a patient's face in a small window, and displays a hangup icon for ending the video call, a mute icon for musting audio, and a video icon for disabling video during the video call;
a vital-measurement icon displayed by the patient user interface;
a vitals measurement processor, activated when the vital-measurement icon is selected;
an Artificial Intelligence (AI) engine having a neural network that is trained to process the real-time video of the patient's face to detect blood flow characteristics that vary over time in the patient's face, the vitals measurement processor generating a vital-sign measurement using the AI engine; and
an augmenter that sends the vital-sign measurement to the clinician after the vitals measurement processor has generated the vital-sign measurement from the real-time video of the patient's face captured during the video call.

17. The computer-program product of claim 16 wherein the computer-readable program code means in the computer-program product further comprises:
a switcher that switches the real-time video of the patient's face to at least one AI engine in the plurality of AI engines in the vitals measurement processor and pauses sending the real-time video of the patient's face to a clinician user interface when the vitals measurement processor has been activated by selecting the vital-measurement icon;
wherein the clinician user interface displays a synthetic image or a still frame from the real-time video of the patient's face when the switcher pauses video during vitals measurement.

18. The computer-program product of claim 16 wherein the patient user interface does not display the real-time video of the clinician when the vitals measurement processor is generating the vital-sign measurement;
wherein audio continues to be exchanged between a clinician user interface and the patient user interface when the vitals measurement processor is generating the vital-sign measurement and video is paused.

19. The computer-program product of claim 16 wherein the computer-readable program code means in the computer-program product further comprises:
a VPC server application, running on a server, the VPC server application comprising:
a vitals service having a plurality of AI engines for use by the vitals measurement processor when a patient device has insufficient computing resources;
a patient records database for storing the vital-sign measurement linked to a record for the patient; and
a bi-directional real-time communication service for connecting a clinician user interface to the patient user interface for the video call.

20. The computer-program product of claim 16 wherein the AI engine further comprises a neural network that is trained to perform Photoplethysmography (PPG), remote Photoplethysmography (rPPG), or Transdermal Optical Imaging (TOI) on the real-time video of the patient's face to generate the vital-sign measurement that selected from the group consisting of a pulse rate, systolic blood pressure, diastolic blood pressure, blood oxygen saturation, and respiration rate.
